(19) United States
(12) Patent Application Publication
(10) Pub. No.: US 2025/0271376 A1
(43) Pub. Date: Aug. 28, 2025

(54) METHODS AND SYSTEMS FOR GENERATING RECOMMENDATIONS BASED UPON BIOELECTRICAL IMPEDANCE ANALYSES

(71) Applicant: Bialume Technologies, Inc., Dallas, TX (US)

(72) Inventors: Marlin Keith Cox, II, Juneau, AK (US); James W. Lincoln, II, Fort Worth, TX (US); Michael Robert Liedtke, Detroit, MI (US)

(21) Appl. No.: 19/059,481

(22) Filed: Feb. 21, 2025

Related U.S. Application Data
(60) Provisional application No. 63/556,634, filed on Feb. 22, 2024.

(51) Int. Cl. G01N 27/02; G01N 33/483; G16B 5/00

(52) U.S. Cl. CPC G01N 27/02; G01N 33/483; G16B 5/00

(57) ABSTRACT

A method for generating recommendations based upon bioelectrical impedance analyses includes generating, by a bioelectrical impedance analyzer, a plurality of measurements of a specimen. The method includes generating, by the bioelectrical impedance analyzer, a measurement of a body composition characteristic based upon the plurality of measurements. The method includes generating, by a computing device in communication with the bioelectrical impedance analyzer, a model of the specimen based upon the plurality of measurements and the measurement of the body composition characteristic. The method includes generating, by the computing device, a recommendation regarding whether to purchase the specimen based upon the generated model. The method includes displaying, by the computing device, a report including an identification of the plurality of measurements, an identification of the measurement of the body composition characteristic, and an identification of the recommendation.

This application claims the benefit of U.S. Patent Application Ser. No. 63/556,634, filed on Feb. 22, 2024, entitled “Methods and Systems for Generating Recommendations Based Upon Bioelectrical Impedace Analyses,” which is hereby incorporated by reference.

### BACKGROUND

The disclosure relates to methods for generating bioelectrical impedance analyses and using such analyses in generating recommendations. More particularly, the methods and systems described herein relate to functionality for modifying a user interface to dynamically display ranked search results with alternate results.

Conventional bioimpedance analysis devices are not typically capable of being used to generate multiple measurements and then to transmit those measurements to computing devices capable of generating real-time recommendations regarding measured specimens. Furthermore, conventional devices are not easily reconfigured to take measures at different single or multiple frequencies thus allowing scalable measures that would encompass whole-organism, organ-level, histological (tissue), cytometry, intercellular and extracellular analysis. Single and multiple measures at various frequencies would also include structural (bone and connective tissue), contractile, nervous, adipose, blood, epithelial, and glandular tissues.

### BRIEF DESCRIPTION

In one aspect, a method for generating recommendations based upon bioelectrical impedance analyses includes generating, by a bioelectrical impedance analyzer, a plurality of measurements of a specimen. The method includes generating, by the bioelectrical impedance analyzer, a measurement of a body composition characteristic based upon the plurality of measurements. The method includes generating, by a computing device in communication with the bioelectrical impedance analyzer, a model of the specimen based upon the plurality of measurements and the measurement of the body composition characteristic. The method includes generating, by the computing device, a recommendation regarding whether to purchase the specimen based upon the generated model. The method includes displaying, by the computing device, a report including an identification of the plurality of measurements, an identification of the measurement of the body composition characteristic, and an identification of the recommendation.

In another aspect, a system for generating recommendations based upon bioelectrical impedance analyses includes a bioelectrical impedance analyzer (i) generating a plurality of measurements of a specimen and generating a measurement of a body composition characteristic based upon the plurality of measurements. The system includes a computing device in communication with the bioelectrical impedance analyzer, (i) generating a model of the specimen based upon the plurality of measurements and the measurement of the body composition characteristic, (ii) generating a recommendation regarding whether to purchase the specimen based upon the generated model, and (iii) displaying report including an identification of the plurality of measurements, an identification of the measurement of the body composition characteristic, and an identification of the recommendation.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a block diagram depicting an embodiment of a system for generating bioelectrical impedance analyses and using such analyses in generating recommendations;

FIG. 2 is a flow diagram depicting an embodiment of a method for generating bioelectrical impedance analyses and using such analyses in generating recommendations; and

FIGS. 3A-3C are block diagrams depicting embodiments of computers useful in connection with the methods and systems described herein.

### DETAILED DESCRIPTION

The methods and systems described herein may provide functionality for generating bioelectrical impedance analyses and using such analyses in generating recommendations. The methods and systems described herein may leverage execution of machine learning programs and output (such as predictions) to generate recommendations regarding generated bioelectrical impedance analyses.

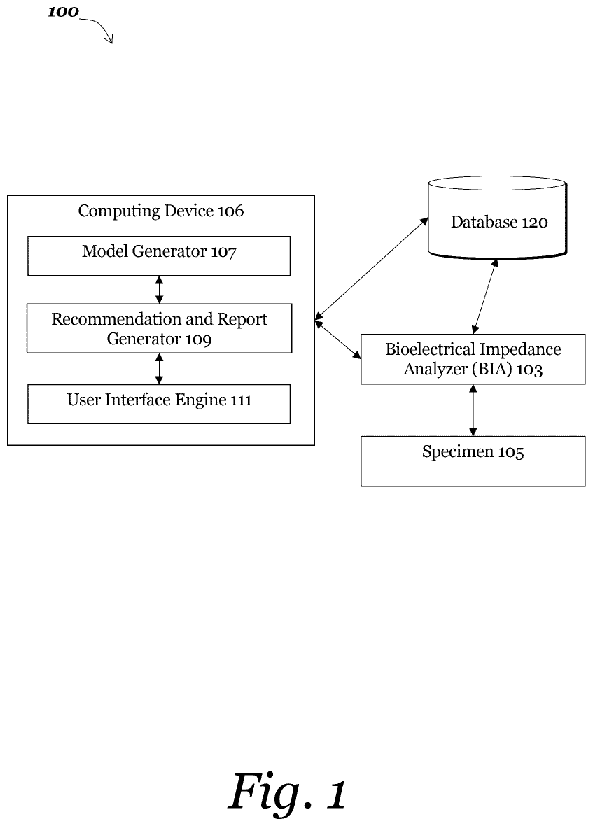

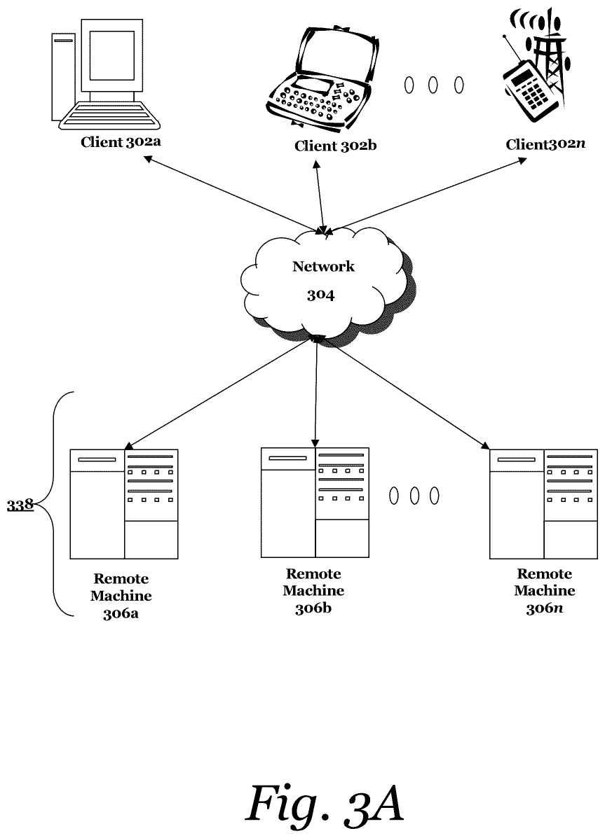

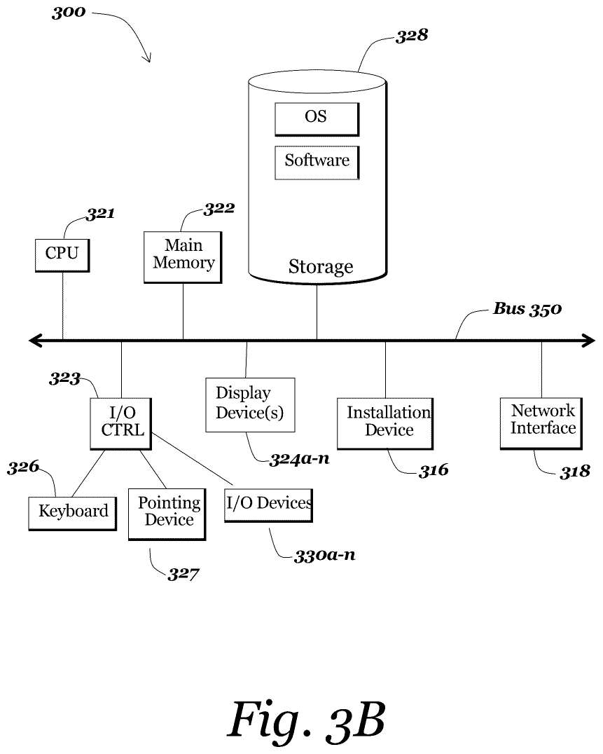

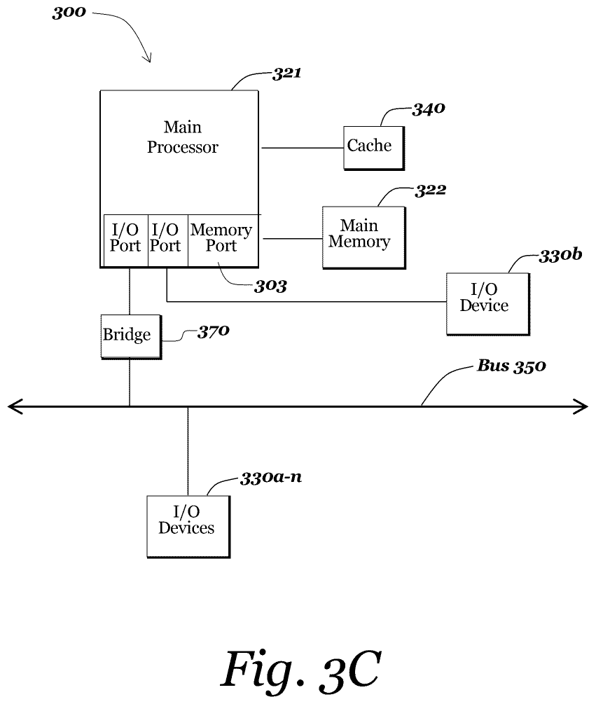

Referring now to FIG. 1, a block diagram depicts one embodiment of a system for generating bioelectrical impedance analyses and using such analyses in generating recommendations. In brief overview, the system 100 includes a bioelectrical impedance analyzer 103 (BIA 103), a specimen 105, a computing device 106, a model generator 107, a recommendation and report generator 109, a user interface engine 111, and a database 120. The computing device 106 may be a modified type or form of computing device (as described in greater detail below in connection with FIGS. 3A-3C) that has been modified to execute the model generator 107, the recommendation and report generator 109, and the user interface engine 111 and to execute instructions for providing the functionality described herein. These modifications result in a new type of computing device that provides a technical solution to problems rooted in computer technology and the field of technological analysis of specimens, including real-time communication with a BIA 103 to receive specimen measurements and generate recommendations regarding the specimen.

The BIA 103 generates a plurality of measurements of a specimen and generates a measurement of a body composition characteristic based upon the plurality of measurements. The BIA 103 may be provided as a software component embedded in a physical device. The BIA 103 may be provided as a hardware component. The BIA 103 may be a handheld device. The BIA 103 may include a connecting structure configured to enable a physical connection between the BIA 103 and a conveyor belt; the BIA 103 may therefore be embedded within a conveyor belt. Therefore, the BIA 103 may generate the plurality of measurements of the specimen while the specimen, on the conveyor belt, passes under the BIA 103. The BIA 103 may be configured to enable a physical connection between the BIA 103 and a band, spring, belt, needle, stripe, or other physical component. The BIA 103 may be configured to enable a physical connection with a joystick, button, foot lever, and/or other form of toggle. The BIA 103 may be a closed loop BIA system where measurements are generated by putting a sample on a scale or by putting electrodes on to the sample. The computing device 106 may be in communication with the BIA 103. Furthermore, autonomous measures are achievable by communication from the computing device 106 and may include periodic measures or event triggered measures by the BIA 103 electrodes being incorporated into the subject invasively (e.g., via wires or needles) or non-invasively

The BIA 103 may be an in-line device with at least one electrode mounted on the BIA 103 device. The BIA 103 may be an automated device. The BIA 103 may include one or more local and/or remote electrodes. At least one electrode may be mounted on a top surface of the BIA 103. The at least one electrode may be mounted on a bottom surface of the BIA 103. The BIA 103 may include functionality for using the electrode to generate one or more measurements of a specimen. The electrodes and the BIA 103 may be stationary while a specimen moves past the BIA 103. Alternatively, or in addition, BIA 103 and any electrodes on the BIA 103 may be configured to be movable and may be moved over and scan the specimen. The BIA 103 may be configured to use different frequencies for different types of specimens. The BIA 103 device may use Bluetooth to transfer data to an application, such as application executed by the computing device 106 or by a computing device of a user of the BIA 103. The BIA 103 may include functionality for transferring data to a cloud database or other cloud-component in a cloud-based solution.

The system 100 may include a camera with which to generate a photograph of a specimen. As an example, the BIA 103 may include the camera. As another example, the BIA 103 may be in communication with a camera independently generating the photograph; in such an embodiment, the BIA 103 may include a receiver for receiving the photograph of the specimen from the camera. As another example, the computing device 106 may be in communication with a camera independently generating the photograph and the BIA 103 is not involved in photographing the specimen or in communication with the camera. As one example, analyzing the photograph may allow the system 100 to add volume metrics to impedance measurements to improve the predictive models used by the system 100 to make recommendations regarding the specimen; therefore, the computing device 106 may include an analyzer generating at least one analysis of at least one characteristic of the photograph of the specimen. As a further example, analyzing the photograph may allow the system 100 to determine volume, length, and weight predictions and to utilize these predictions in conjunction with BIA 103 measurements to enhance predictive accuracy. In some embodiments, the computing device 106 may include a receiver for receiving the photograph of the specimen from the camera and an analyzer generating at least one analysis of at least one characteristic of the photograph of the specimen.

The system 100 may use one or more biosensors in addition to the BIA 103. A biosensor may be a device that combines a biological recognition element (such as enzymes, antibodies, or receptors) with a transducer (a component that converts a biochemical signal into a measurable electrical, optical, or physical signal). Biosensors may be designed to detect and quantify specific biological molecules or physiological parameters. Biosensors can be invasive or non-invasive. Biosensors may provide functionality allowing the BIA 103 to interact with an analyte, causing a signal generation and subsequent detection which may be output via the system 100. The BIA 103 may be modified to include or communicate with such biosensors, providing novel functionality.

In some embodiments, the BIA 103 may be a device providing functionality for changing biosensor attributes to meet different measuring possibilities. The BIA 103 may include four electrodes for measuring seafood. The BIA 103 may be or include the functionality of an EKG with leads for generate measurements from humans. The BIA 103 may include one or more alligator clip plugs to measure a variety of cellular samples, depending on measuring invasively (needle electrodes) vs non-invasively.

The computing device 106 may for generating the model based upon the plurality of measurements and the measurement of the body composition characteristic and an output of the at least one analysis of the at least one characteristic of the photograph of the specimen.

The model generator 107 may be provided as a software component. The model generator 107 may be provided as a hardware component. The computing device 106 may be in communication with the model generator 107. The computing device 106 may execute the model generator 107. In embodiments in which the system 100 includes a camera, the computing device 106 may include functionality for receiving the photograph of the specimen from the camera and may include an analyzer generating at least one analysis of at least one characteristic of the photograph of the specimen. The model generator 107 may include functionality for generating the model of the specimen based upon the plurality of measurements and the measurement of the body composition characteristic and the at least one analysis of the at least one characteristic of the photograph of the specimen.

In one embodiment, the recommendation and report generator 109 is implemented as a software component. In another embodiment, the recommendation and report generator 109 is implemented as a hardware component. The computing device 106 may be in communication with the recommendation and report generator 109. The computing device 106 may execute the recommendation and report generator 109.

In one embodiment, the user interface engine 111 may be provided as a software component. In another embodiment, the user interface engine 111 may be provided as a hardware component. The computing device 106 may be in communication with the user interface engine 111. The computing device 106 may execute the user interface engine 111. The user interface engine 111 may provide functionality for receiving data from the recommendation and report generator 109. The user interface engine 111 may provide functionality for generating user interfaces displayed to a user of the BIA 103. The user interface engine 111 may provide functionality for modifying data displayed within user interfaces displayed to a user of the BIA 103.

The computing device 106 may include or be in communication with the database 120. The database 120 may store data related to BIA measurements, specimen data, and previously generated recommendations and reports. The database 120 may be an ODBC-compliant database. For example, the database 120 may be provided as an ORACLE database, manufactured by Oracle Corporation of Redwood Shores, CA. In other embodiments, the database 120 can be a Microsoft ACCESS database or a Microsoft SQL server database, manufactured by Microsoft Corporation of Redmond, WA. In other embodiments, the database 120 can be a SQLite database distributed by Hwaci of Charlotte, NC, or a PostgreSQL database distributed by The PostgreSQL Global Development Group. In still other embodiments, the database 120 may be a custom-designed database based on an open source database, such as the MYSQL family of freely available database products distributed by Oracle Corporation of Redwood City, CA. In other embodiments, examples of databases include, without limitation, structured storage (e.g., NoSQL-type databases and BigTable databases), HBase databases distributed by The Apache Software Foundation of Forest Hill, MD, MongoDB databases distributed by 10Gen, Inc., of New York, NY, an AWS DynamoDB distributed by Amazon Web Services and Cassandra databases distributed by The Apache Software Foundation of Forest Hill, MD. In further embodiments, the database 120 may be any form or type of database.

Although, for ease of discussion, the BIA 103, the model generator 107, the recommendation and report generator 109, the user interface engine 111, the computing device 106, and the database 120 are described in FIG. 1 as separate modules, it should be understood that this does not restrict the architecture to a particular implementation. For instance, these components may be encompassed by a single circuit or software function or, alternatively, distributed across a plurality of computing devices.

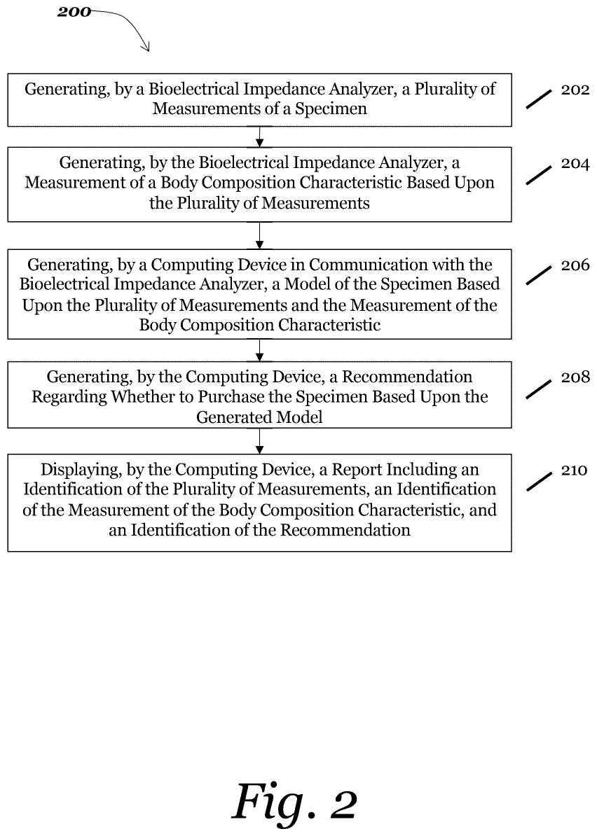

Referring now to FIG. 2, in brief overview, a block diagram depicts one embodiment of a method 200 for generating bioelectrical impedance analyses and using such analyses in generating recommendations. The method 200 includes generating, by a bioelectrical impedance analyzer, a plurality of measurements of a specimen (202). The method 200 includes generating, by the bioelectrical impedance analyzer, a measurement of a body composition characteristic based upon the plurality of measurements (204). The method 200 includes generating, by a computing device in communication with the bioelectrical impedance analyzer, a model of the specimen based upon the plurality of measurements and the measurement of the body composition characteristic (206). The method 200 includes generating, by the computing device, a recommendation regarding whether to purchase the specimen based upon the generated model (208). The method 200 includes displaying, by the computing device, a report including an identification of the plurality of measurements, an identification of the measurement of the body composition characteristic, and an identification of the recommendation (210).

Referring now to FIG. 2, in greater detail and in connection with FIG. 1, the method 200 includes generating, by a bioelectrical impedance analyzer, a plurality of measurements of a specimen (202). The bioelectrical impedance analyzer may be the BIA 103. The plurality of measures may include a measurement of a weight of the specimen. The plurality of measures may include a measurement of a length of the specimen. The plurality of measures may include a measurement of a rate at which an electrical current travels through the specimen.

In embodiments in which the BIA 103 includes a connecting structure configured to enable the BIA 103 to be connected to a conveyor belt, as described above in connection with FIG. 1, the BIA 103 may generate the plurality of measurements of the specimen while the specimen, on the conveyor belt, passes under the BIA 103. In other embodiments, the BIA 103 may generate the plurality of measurements of the specimen while the BIA 103 passes over the specimen.

The BIA 103 may generate measurements of factors including, without limitation, salt content, water movement, salt concentration changes, liquid brine strengths, electrolyte fluctuations, protein denaturation, cell membrane integrity, sugar content, volumetric changes, muscle contractions, rigor mortis and body composition changes. The changes of these components are reflective of the specimen or organism being measured.

The method 200 includes generating, by the bioelectrical impedance analyzer, a measurement of a body composition characteristic based upon the plurality of measurements (204). In some embodiments, one or more electrical equations used by the BIA 103 represent different circuits within the system and include conductance, impedance, phase shifts, reactance, capacitance and is available in two (fat mass and fat free mass), three compartments (addition of hydration), and segmental (specific regions such as arm or leg). Different circuit combinations paired with different frequencies may provide predictive modeling capabilities of different body composition characteristics. Specific results may be generated by components of different form factors (e.g., probes, electrodes, etc.) and frequencies. The measurements of the body composition characteristic may represent tissue characteristics. The measurements of the body composition characteristic may represent specimen characteristics.

The method 200 includes generating, by a computing device in communication with the bioelectrical impedance analyzer, a model of the specimen based upon the plurality of measurements and the measurement of the body composition characteristic (206). Generated measures from the BIA 103 may be used in different electrical circuit representations with different frequencies representing different circuits and may be characterized as dependent variables. The dependent variable circuitries may be modeled against known values of interest which are characterized as independent variables. Predictive models may then used to estimate those independent parameters and/or to detect change in them. The BIA 103 may be a statistical measuring tool that uses objective cellular inputs. A novel unique formula is created through a third-party study (using a single frequency and multi-frequency approach) and then the statistical results are aggregated and turned into a formula. The BIA 103 may use multiple frequencies in generating specimen measurements to obtain a line signature; the entire line may be used to define at least one characteristic of the specimen and how the line changes may represent how the specimen changes. By using multiple frequencies and moving the specimen while the measurements are being generated, the BIA 103 may identify isolated areas of disease within the specimen. Analyzing the photograph may allow the system 100 to determine volume, length, and weight predictions and to utilize these predictions in conjunction with BIA 103 measurements to enhance predictive accuracy.

The method 200 may include generating a photograph of the specimen. In embodiments in which the system 100 includes a camera (as either an independent device or as a component of the BIA 103) and the computing device 106 receives a photograph of the specimen, the computing device 106 may analyze (directly or via execution of the model generator 107) at least one characteristic of the photograph (and, therefore, at least one characteristic of the specimen). The model generator 107 may then generate and/or update the model of the specimen based upon the plurality of measurements, and the measurement of the body composition characteristic, and the analysis of the at least one characteristic of the photograph of the specimen.

The method 200 includes generating, by the computing device, a recommendation regarding whether to purchase the specimen based upon the generated model (208). The predictive model for a set-independent variable may provide estimates or detect changes in a selected parameter. The provided estimates may be used within a set of rules that are standard or in relation to a feedback system from external inputs. Rules may be dynamic and can be regenerated based on inputs from the BIA 103, external inputs and/or internal estimate generations.

The method 200 may include generating a real-time recommendation relating to a variety of determinations, such as, without limitation, whether the specimen is healthy, whether the specimen is ripe or otherwise ready for processing (e.g., when the specimen is a fruit or vegetable), whether the specimen is fertilized (e.g., for eggs), a level of doneness of the specimen (e.g., whether an egg is hard boiled or soft boiled), muscle myopathies, fluid movement, and intracellular and extracellular solute concentrations. The method 200 may include generating a real-time recommendation regarding an amount to pay for the specimen based on analyses of one or more of the measurements of the specimen (e.g., recommending a price based on a level of quality). The method 200 may include generating a real-time recommendation including data supporting decisions based on predicted metrics, resulting in an action by the user.

The method 200 includes displaying, by the computing device, a report including an identification of the plurality of measurements, an identification of the measurement of the body composition characteristic, and an identification of the recommendation (210). The user interface engine 111 may generate the report. The user interface engine 111 may generate a report incorporating some or all of the measurements of any kind. The user interface engine 111 may generate a report incorporating some or all of the analyses of any of the measurements. The user interface engine 111 may generate a report incorporating some or all of the generated recommendations. For example, the user interface engine 111 may generate a report incorporating recommendations regarding whether or not to purchase a specimen to a user that is a buyer while the user interface engine 111 may generate a report incorporating recommendations regarding whether or not a specimen is ripe or otherwise ready for non-buyer users. Users may also include practitioners, individuals, or others using the analytics to make a decision and act upon that decision using the data.

The method 200 may include execution of a machine learning engine (not shown in FIG. 1) to analyze the specimen measurements and generate improved recommendations. Execution of the optional machine learning engine may result in identification of new factors or co-factors to incorporate into subsequent analyses. Execution of the optional machine learning engine may result in identification of meta-data to be gathered prior to taking subsequent measurements and which may be incorporated into the recommendation generation process resulting in improved recommendations. For example, a machine learning engine may analyze existing bioelectrical equations, frequencies and combinations of both to determine when changes in factors and co-factors and the influence of entered meta-data affect impedance measurement change and determine what recommendations to suggest as a result. As a further example, in an embodiment in which the specimen is a chicken egg, the method 200 may include executing a machine learning engine that receives the measurements generated about the specimen and determine which measurement frequency the BIA 103 should use in generating subsequent measurements and which bioelectrical equations to apply to more accurately determine whether the chicken egg has been fertilized; such improvements could result in the recommendation and report generator 109 determining not only whether the chicken egg has been fertilized but to determine when the egg was fertilized and to generate one or more recommendations regarding what to do with fertilized eggs or with unfertilized eggs (e.g., storage options, options regarding whether or not to purchase the eggs, options regarding variable pricing of the eggs for different purposes, and so on). Execution of the machine learning engine may include execution of R-statistical software. Execution of the machine learning engine may include execution of programs such as MATLAB and SAS.

In some embodiments, the BIA 103 generates measurements “in situ” or substantially within a natural environment of interest. For example, the BIA 103 may include an electrode measuring device for generating measurements in aquatic environments. Aquatic environments may include underwater environments like rivers, lakes, oceans, or other aquatic systems. Conducting in situ bioimpedance measurements underwater may provide valuable insights into the physiological responses of organisms and ecosystems to their specific aquatic habitats.

In situ bioimpedance measurements underwater could be used for various applications, including without limitation environmental monitoring, behavioral studies, physiological research, biomedical applications, and remote sensing. Bioimpedance measurements of aquatic organisms in their natural habitat can provide information about their health, stress levels, and hydration status; his data can be used to assess the overall health of aquatic ecosystems and detect potential environmental changes or pollution. Monitoring the bioimpedance of aquatic animals in situ may offer insights into their behavior, movements, and responses to different environmental conditions. In situ bioimpedance measurements may be part of studies aimed at understanding the physiological adaptations of aquatic organisms to their environment; researchers can analyze how these adaptations influence their impedance profiles. In situ bioimpedance measurements may be used in medical research related to aquatic organisms, such as fish or marine mammals; these measurements might be used to monitor the health of captive animals or assess the impacts of various factors on their well-being. In situ bioimpedance measurements may be integrated into remote sensing systems to provide real-time data on aquatic environments; this functionality may be used for monitoring changes in water quality or detecting anomalies in underwater ecosystems. Impedance detection may be used to detect microbes in an aquatic environment. Impedance detection may be used to generate water quality metrics in an aquatic environment. Given the challenges of conducting bioimpedance measurements underwater, the system 100 may include specialized equipment and/or may apply specialized techniques. These might include waterproof electrodes, measurement protocols, and advanced data processing methods to account for the unique characteristics of underwater environments.

In some embodiments, the system 100 includes non-transitory, computer-readable medium comprising computer program instructions tangibly stored on the non-transitory computer-readable medium, wherein the instructions are executable by at least one processor to perform each of the steps described above in connection with FIG. 1

It should be understood that the systems described above may provide multiple ones of any or each of those components and these components may be provided on either a standalone machine or, in some embodiments, on multiple machines in a distributed system. The phrases ‘in one embodiment,’ ‘in another embodiment,’ and the like, generally mean that the particular feature, structure, step, or characteristic following the phrase is included in at least one embodiment of the present disclosure and may be included in more than one embodiment of the present disclosure. Such phrases may, but do not necessarily, refer to the same embodiment. However, the scope of protection is defined by the appended claims; the embodiments mentioned herein provide examples.

The terms “A or B”, “at least one of A or/and B”, “at least one of A and B”, “at least one of A or B”, or “one or more of A or/and B” used in the various embodiments of the present disclosure include any and all combinations of words enumerated with it. For example, “A or B”, “at least one of A and B” or “at least one of A or B” may mean (1) including at least one A, (2) including at least one B, (3) including either A or B, or (4) including both at least one A and at least one B.

Any step or act disclosed herein as being performed, or capable of being performed, by a computer or other machine, may be performed automatically by a computer or other machine, whether or not explicitly disclosed as such herein. A step or act that is performed automatically is performed solely by a computer or other machine, without human intervention. A step or act that is performed automatically may, for example, operate solely on inputs received from a computer or other machine, and not from a human. A step or act that is performed automatically may, for example, be initiated by a signal received from a computer or other machine, and not from a human. A step or act that is performed automatically may, for example, provide output to a computer or other machine, and not to a human.

Although terms such as “optimize” and “optimal” may be used herein, in practice, embodiments of the present invention may include methods which produce outputs that are not optimal, or which are not known to be optimal, but which nevertheless are useful. For example, embodiments of the present invention may produce an output which approximates an optimal solution, within some degree of error. As a result, terms herein such as “optimize” and “optimal” should be understood to refer not only to processes which produce optimal outputs, but also processes which produce outputs that approximate an optimal solution, within some degree of error.

The systems and methods described above may be implemented as a method, apparatus, or article of manufacture using programming and/or engineering techniques to produce software, firmware, hardware, or any combination thereof. The techniques described above may be implemented in one or more computer programs executing on a programmable computer including a processor, a storage medium readable by the processor (including, for example, volatile and non-volatile memory and/or storage elements), at least one input device, and at least one output device. Program code may be applied to input entered using the input device to perform the functions described and to generate output. The output may be provided to one or more output devices.

Each computer program within the scope of the claims below may be implemented in any programming language, such as assembly language, machine language, a high-level procedural programming language, or an object-oriented programming language. The programming language may, for example, be LISP, PROLOG, PERL, C, C++, C#, JAVA, Python, Rust, Go, or any compiled or interpreted programming language.

Each such computer program may be implemented in a computer program product tangibly embodied in a machine-readable storage device for execution by a computer processor. Method steps may be performed by a computer processor executing a program tangibly embodied on a computer-readable medium to perform functions of the methods and systems described herein by operating on input and generating output. Suitable processors include, by way of example, both general and special purpose microprocessors. Generally, the processor receives instructions and data from a read-only memory and/or a random access memory. Storage devices suitable for tangibly embodying computer program instructions include, for example, all forms of computer-readable devices, firmware, programmable logic, hardware (e.g., integrated circuit chip; electronic devices; a computer-readable non-volatile storage unit; non-volatile memory, such as semiconductor memory devices, including EPROM, EEPROM, and flash memory devices; magnetic disks such as internal hard disks and removable disks; magneto-optical disks; and CD-ROMs). Any of the foregoing may be supplemented by, or incorporated in, specially-designed ASICs (application-specific integrated circuits) or FPGAs (Field-Programmable Gate Arrays). A computer can generally also receive programs and data from a storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium. A computer may also receive programs and data (including, for example, instructions for storage on non-transitory computer-readable media) from a second computer providing access to the programs via a network transmission line, wireless transmission media, signals propagating through space, radio waves, infrared signals, etc.

Referring now to FIGS. 3A, 3B, and 3C, block diagrams depict additional detail regarding computing devices that may be modified to execute novel, non-obvious functionality for implementing the methods and systems described above.

Referring now to FIG. 3A, an embodiment of a network environment is depicted. In brief overview, the network environment comprises one or more clients 302a-302n (also generally referred to as local machine(s) 302, client(s) 302, client node(s) 302, client machine(s) 302, client computer(s) 302, client device(s) 302, computing device(s) 302, endpoint(s) 302, or endpoint node(s) 302) in communication with one or more remote machines 306a-306n (also generally referred to as server(s) 306 or computing device(s) 306) via one or more networks 304.

Although FIG. 3A shows a network 304 between the clients 302 and the remote machines 306, the clients 302 and the remote machines 306 may be on the same network 304. The network 304 can be a local area network (LAN), such as a company Intranet, a metropolitan area network (MAN), or a wide area network (WAN), such as the Internet or the World Wide Web. In some embodiments, there are multiple networks 304 between the clients 302 and the remote machines 306. In one of these embodiments, a network 304′ (not shown) may be a private network and a network 304 may be a public network. In another of these embodiments, a network 304 may be a private network and a network 304′ a public network. In still another embodiment, networks 304 and 304′ may both be private networks. In yet another embodiment, networks 304 and 304′ may both be public networks.

The network 304 may be any type and/or form of network and may include any of the following: a point to point network, a broadcast network, a wide area network, a local area network, a telecommunications network, a data communication network, a computer network, an ATM (Asynchronous Transfer Mode) network, a SONET (Synchronous Optical Network) network, an SDH (Synchronous Digital Hierarchy) network, a wireless network, a wireline network, an Ethernet, a virtual private network (VPN), a software-defined network (SDN), a network within the cloud such as AWS VPC (Virtual Private Cloud) network or Azure Virtual Network (VNet), and a RDMA (Remote Direct Memory Access) network. In some embodiments, the network 304 may comprise a wireless link, such as an infrared channel or satellite band. The topology of the network 304 may be a bus, star, or ring network topology. The network 304 may be of any such network topology as known to those ordinarily skilled in the art capable of supporting the operations described herein. The network may comprise mobile telephone networks utilizing any protocol or protocols used to communicate among mobile devices (including tables and handheld devices generally), including AMPS, TDMA, CDMA, GSM, GPRS, UMTS, or LTE. In some embodiments, different types of data may be transmitted via different protocols. In other embodiments, the same types of data may be transmitted via different protocols.

A client 302 and a remote machine 306 (referred to generally as computing device 300 or as machines 300) can be any workstation, desktop computer, laptop or notebook computer, server, portable computer, mobile telephone, mobile smartphone, or other portable telecommunication device, media playing device, a gaming system, mobile computing device, or any other type and/or form of computing, telecommunications or media device that is capable of communicating on any type and form of network and that has sufficient processor power and memory capacity to perform the operations described herein. A client 302 may execute, operate or otherwise provide an application, which can be any type and/or form of software, program, or executable instructions, including, without limitation, any type and/or form of web browser, web-based client, client-server application, an ActiveX control, a JAVA applet, a webserver, a database, an HPC (high performance computing) application, a data processing application, or any other type and/or form of executable instructions capable of executing on client 302.

In one embodiment, a computing device 306 provides functionality of a web server. The web server may be any type of web server, including web servers that are open-source web servers, web servers that execute proprietary software, and cloud-based web servers where a third party hosts the hardware executing the functionality of the web server. In some embodiments, a web server 306 comprises an open-source web server, such as the APACHE servers maintained by the Apache Software Foundation of Delaware. In other embodiments, the web server executes proprietary software, such as the INTERNET INFORMATION SERVICES products provided by Microsoft Corporation of Redmond, WA, the ORACLE IPLANET web server products provided by Oracle Corporation of Redwood Shores, CA, or the ORACLE WEBLOGIC products provided by Oracle Corporation of Redwood Shores, CA.

In some embodiments, the system may include multiple, logically-grouped remote machines 306. In one of these embodiments, the logical group of remote machines may be referred to as a server farm 338. In another of these embodiments, the server farm 338 may be administered as a single entity.

FIGS. 3B and 3C depict block diagrams of a computing device 400 useful for practicing an embodiment of the client 302 or a remote machine 306. As shown in FIGS. 3B and 3C, each computing device 300 includes a central processing unit 321, and a main memory 322. As shown in FIG. 3B, a computing device 300 may include a storage device 328, an installation device 316, a network interface 318, an I/O controller 323, display devices 324a-n, a keyboard 326, a pointing device 327, such as a mouse, and one or more other I/O devices 330a-n. The storage device 328 may include, without limitation, an operating system and software. As shown in FIG. 3C, each computing device 300 may also include additional optional elements, such as a memory port 303, a bridge 370, one or more input/output devices 330a-n (generally referred to using reference numeral 330), and a cache memory 340 in communication with the central processing unit 321.

The central processing unit 321 is any logic circuitry that responds to and processes instructions fetched from the main memory 322. In many embodiments, the central processing unit 321 is provided by a microprocessor unit, such as: those manufactured by Intel Corporation of Mountain View, CA; those manufactured by Motorola Corporation of Schaumburg, IL; those manufactured by Transmeta Corporation of Santa Clara, CA; those manufactured by International Business Machines of White Plains, NY; or those manufactured by Advanced Micro Devices of Sunnyvale, CA. Other examples include RISC-V processors, SPARC processors, ARM processors, processors used to build UNIX/LINUX “white” boxes, and processors for mobile devices. The computing device 300 may be based on any of these processors, or any other processor capable of operating as described herein.

Main memory 322 may be one or more memory chips capable of storing data and allowing any storage location to be directly accessed by the central processing unit 321. The main memory 322 may be based on any available memory chips capable of operating as described herein. In the embodiment shown in FIG. 3B, the central processing unit 321 communicates with main memory 322 via a system bus 350. FIG. 3C depicts an embodiment of a computing device 300 in which the central processing unit 321 communicates directly with main memory 322 via a memory port 303. FIG. 3C also depicts an embodiment in which the central processing unit 321 communicates directly with cache memory 340 via a secondary bus, sometimes referred to as a backside bus. In other embodiments, the central processing unit 321 communicates with cache memory 340 using the system bus 350.

In the embodiment shown in FIG. 3B, the central processing unit 321 communicates with various I/O devices 330 via a local system bus 350. Various buses may be used to connect the central processing unit 321 to any of the I/O devices 330, including a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display device 324, the central processing unit 321 may use an Advanced Graphics Port (AGP) to communicate with the display device 324. FIG. 3C depicts an embodiment of a computing device 300 in which the central processing unit 321 also communicates directly with an I/O device 330b via, for example, HYPERTRANSPORT, RAPIDIO, or INFINIBAND communications technology.

One or more of a wide variety of I/O devices 330a-n may be present in or connected to the computing device 300, each of which may be of the same or different type and/or form. Input devices include keyboards, mice, trackpads, joysticks, buttons, foot levers, trackballs, microphones, scanners, cameras, toggles, and drawing tablets. Output devices include video displays, speakers, inkjet printers, laser printers, 3D printers, and dye-sublimation printers. The I/O devices may be controlled by an I/O controller 323 as shown in FIG. 3B. Furthermore, an I/O device may also provide storage and/or an installation device 316 for the computing device 300. In some embodiments, the computing device 300 may provide USB connections (not shown) to receive handheld USB storage devices such as the USB Flash Drive line of devices manufactured by Twintech Industry, Inc. of Los Alamitos, CA.

Referring still to FIG. 3B, the computing device 400 may support any suitable installation device 316, such as hardware for receiving and interacting with removable storage; e.g., disk drives of any type, CD drives of any type, DVD drives, tape drives of various formats, USB devices, external hard drives, or any other device suitable for installing software and programs. In some embodiments, the computing device 300 may provide functionality for installing software over a network 304. The computing device 300 may further comprise a storage device, such as one or more hard disk drives or redundant arrays of independent disks, for storing an operating system and other software. Alternatively, the computing device 300 may rely on memory chips for storage instead of hard disks.

Furthermore, the computing device 300 may include a network interface 318 to interface to the network 304 through a variety of connections including, but not limited to, standard telephone lines, LAN or WAN links (e.g., 802.11, T1, T3, 56 kb, X.25, SNA, DECNET, RDMA), broadband connections (e.g., ISDN, Frame Relay, ATM, Gigabit Ethernet, Ethernet-over-SONET), wireless connections, virtual private network (VPN) connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, 802.15.4, Bluetooth, ZIGBEE, CDMA, GSM, WiMax, and direct asynchronous connections). In one embodiment, the computing device 300 communicates with other computing devices 300′ via any type and/or form of gateway or tunneling protocol such as GRE, VXLAN, IPIP, SIT, ip6tnl, VTI and VTI6, IP6GRE, FOU, GUE, GENEVE, ERSPAN, Secure Socket Layer (SSL) or Transport Layer Security (TLS). The network interface 318 may comprise a built-in network adapter, network interface card, PCMCIA network card, card bus network adapter, wireless network adapter, USB network adapter, modem, or any other device suitable for interfacing the computing device 300 to any type of network capable of communication and performing the operations described herein.

In further embodiments, an I/O device 330 may be a bridge between the system bus 350 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a HIPPI bus, a Super HIPPI bus, a Serial Plus bus, a SCI/LAMP bus, a Fibre Channel bus, or a Serial Attached small computer system interface bus.

A computing device 300 of the sort depicted in FIGS. 3B and 3C typically operates under the control of operating systems, which control scheduling of tasks and access to system resources. The computing device 300 can be running any operating system such as any of the versions of the MICROSOFT WINDOWS operating systems, the different releases of the UNIX and LINUX operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 7, WINDOWS 8, WINDOWS VISTA, WINDOWS 10, and WINDOWS 11 all of which are manufactured by Microsoft Corporation of Redmond, WA; MAC OS manufactured by Apple Inc. of Cupertino, CA; OS/2 manufactured by International Business Machines of Armonk, NY; Red Hat Enterprise Linux, a Linux-variant operating system distributed by Red Hat, Inc., of Raleigh, NC; Ubuntu, a freely-available operating system distributed by Canonical Ltd. of London, England; CentOS, a freely-available operating system distributed by the centos.org community; SUSE Linux, a freely-available operating system distributed by SUSE, or any type and/or form of a Unix operating system, among others.

Having described certain embodiments of methods and systems for generating bioelectrical impedance analyses and using such analyses in generating recommendations, it will be apparent to one of skill in the art that other embodiments incorporating the concepts of the disclosure may be used. Therefore, the disclosure should not be limited to certain embodiments but rather should be limited only by the spirit and scope of the following claims.

What is claimed is:

1. A method for generating recommendations based upon bioelectrical impedance analyses, the method comprising:
generating, by a bioelectrical impedance analyzer, a plurality of measurements of a specimen;
generating, by the bioelectrical impedance analyzer, a measurement of a body composition characteristic based upon the plurality of measurements;
generating, by a computing device in communication with the bioelectrical impedance analyzer, a model of the specimen based upon the plurality of measurements and the measurement of the body composition characteristic;
generating, by the computing device, a recommendation regarding whether to purchase the specimen based upon the generated model; and
displaying, by the computing device, a report including an identification of the plurality of measurements, an identification of the measurement of the body composition characteristic, and an identification of the recommendation.

2. The method of claim 1 wherein generating the plurality of measurements further comprises generating a measurement of a weight of the specimen.

3. The method of claim 1 wherein generating the plurality of measurements further comprises generating a measurement of a length of the specimen.

4. The method of claim 1 wherein generating the plurality of measurements further comprises generating a measurement of a rate at which an electrical current travels through the specimen.

5. The method of claim 1 further comprising generating, by a camera, a photograph of the specimen.

6. The method of claim 5 further comprising analyzing, by the computing device in communication with the bioelectrical impedance analyzer, at least one characteristic of the photograph.

7. The method of claim 6 wherein generating the model of the specimen further comprises generating the model based upon the plurality of measurements and the measurement of the body composition characteristic and an output of the analyzing of the at least one characteristic of the photograph of the specimen.

8. A system comprising:
a bioelectrical impedance analyzer (i) generating a plurality of measurements of a specimen and generating a measurement of a body composition characteristic based upon the plurality of measurements; and
a computing device in communication with the bioelectrical impedance analyzer, (i) generating a model of the specimen based upon the plurality of measurements and the measurement of the body composition characteristic, (ii) generating a recommendation regarding whether to purchase the specimen based upon the generated model, and (iii) displaying report including an identification of the plurality of measurements, an identification of the measurement of the body composition characteristic, and an identification of the recommendation.

9. The system of claim 8, wherein the bioelectrical impedance analyzer is a handheld device.

10. The system of claim 8, wherein the bioelectrical impedance analyzer further comprises a connecting structure for connecting to a conveyor belt and wherein the bioelectrical impedance analyzer generates the plurality of measurements of the specimen while the specimen, on the conveyor belt, passes under the bioelectrical impedance analyzer.

11. The system of claim 8, wherein the bioelectrical impedance analyzer is embedded within a conveyor belt and executes to generate the plurality of measurements of the specimen while the specimen, on the conveyor belt, passes under the bioelectrical impedance analyzer.

12. The system of claim 8 further comprising a camera generating a photograph of the specimen.

13. The system of claim 12, wherein the computing device further comprises:
a receiver for receiving the photograph of the specimen from the camera; and
and an analyzer generating at least one analysis of at least one characteristic of the photograph of the specimen.

14. The system of claim 13, wherein the computing device further comprises means for generating the model based upon the plurality of measurements and the measurement of the body composition characteristic and the at least one analysis of the at least one characteristic of the photograph of the specimen.
